(19) United States
(12) Patent Application Publication
(10) Pub. No.: US 2002/0009427 A1
(43) Pub. Date: Jan. 24, 2002

(54) Methods of therapy for non-hodgkin's lymphoma

(76) Inventors: Maurice J. Wolin, Emeryville, CA (US); Joseph D. Rosenblatt, Rochester, NY (US)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 09/815,597

(22) Filed: Mar. 23, 2001

Related U.S. Application Data
(60) Provisional application No. 60/192,047, filed on Mar. 24, 2000.

(51) Int. Cl. A61K 39/395; A61K 38/20

(52) U.S. Cl. 424/85.2; 424/141.1

(57) ABSTRACT

Methods for treating a mammal with lymphoma using a combination of interleukin-2 (IL-2) or variant thereof and at least one anti-CD20 antibody or fragment thereof are provided. These anti-tumor agents are administered as two separate pharmaceutical compositions, one containing IL-2 (or variant thereof), the other containing at least one anti-CD20 antibody (or fragment thereof), according to a dosing regimen. Administering of these two agents together potentiates the effectiveness of either agent alone, resulting in a positive therapeutic response that is improved with respect to that observed with either agent alone. The anti-tumor effects of these agents can be achieved using lower dosages of IL-2, thereby lessening the toxicity of prolonged IL-2 administration and the potential for tumor escape.

### CROSS-REFERENCE TO RELATED APPLICATION

This application claims the benefit of U.S. Provisional Application Ser. No. 60/192,047, filed Mar. 24, 2000, the contents of which are herein incorporated by reference in their entirety.

### FIELD OF THE INVENTION

The present invention is directed to methods of therapy for non-Hodgkin's lymphoma, more particularly to concurrent therapy with interleukin-2 and monoclonal antibodies targeting the CD20 B-cell surface antigen.

### BACKGROUND OF THE INVENTION

The non-Hodgkin's lymphomas are a diverse group of malignancies that are predominately of B-cell origin. In the Working Formulation classification scheme, these lymphomas been divided into low-, intermediate-, and high-grade categories by virtue of their natural histories (see “The Non-Hodgkin's Lymphoma Pathologic Classification Project,” Cancer 49(1982): 2112-2135). The low-grade or favorable lymphomas are indolent, with a median survival of 5 to 10 years (Horning and Rosenberg (1984) N. Engl. J Med. 311:1471-1475). Although chemotherapy can induce remissions in the majority of indolent lymphomas, cures are rare and most patients eventually relapse, requiring further therapy. The intermediate- and high-grade lymphomas are more aggressive tumors, but they have a greater chance for cure with chemotherapy. However, significant numbers of these patients will still relapse and require further treatment.

Interleukin-2 (IL-2) is a potent stimulator of natural killer (NK) and T-cell proliferation and function (Morgan et al. (1976) Science 193:1007-1011). This naturally occurring lymphokine has been shown to have antitumor activity against a variety of malignancies either alone or when combined with leukotriene-activated killer (LAK) cells or tumor-infiltrating lymphocytes (see, for example, Rosenberg et al. (1987) N. Engl. J Med. 316:889-897; Rosenberg (1988) Ann. Surg. 208:121-135; Topalian et al. 1988) J Clin. Oncol. 6:839-853; Rosenberg et al. (1988) N. Engl. J Med. 319:1676-1680; and Weber et al. (1992) J Clin. Oncol. 10:33-40). Although the anti-tumor activity of IL-2 has best been described in patients with metastatic melanoma and renal cell carcinoma, other diseases, notably lymphoma, also appear to respond to treatment with IL-2. However, high doses of IL-2 used to achieve positive therapeutic results with respect to tumor growth frequently cause severe toxicity effects, including capillary leak, hypotension, and nuerological changes (see, for example, Duggan et al. (1992) J Immunotherapy 12:115-122; Gisselbrecht et al (1994) Blood 83:2081-2085; and Sznol and Parkinson 1994) Blood 83:2020-2022).

Cancer research has shown an increasing interest in the use of monoclonal antibodies as a therapeutic. Raised in a similar fashion to diagnostic antibodies, therapeutic antibodies are aimed at specifically targeting tumor cells. The use of therapeutic monoclonal antibodies has been hampered in the past primarily because of issues related to the antigenicity of the protein. Monoclonal antibodies are a mouse product, and therefore generate an anti-murine response when injected into humans. This so-called HAMA (human anti-mouse antibody) response has imposed a great limitation on the use of monoclonal antibodies, as repeated dosing is nearly always precluded. In addition, serious complications, such as serum sickness, have been reported with the use of these agents. With the advent of chimeric and humanized antibodies, the therapeutic benefit of monoclonals is being realized. Using recombinant DNA technology, it is possible for a monoclonal antibody to be constructed by joining the variable or antigen recognition site of the antibody to a human backbone. This construction greatly decreases the incidence of blocking or clearing of the foreign antibodies from the host. This development allows for multiple doses of antibody to be given, providing the opportunity for reproducible and sustained responses with this therapy.

Monoclonal antibodies have increasingly become a method of choice for the treatment of lymphomas of the B-cell type. All B-cells express common cell surface markers, including CD20 and CD 19. CD20 is a 33-37 kDa phosphoprotein that is expressed early in B-cell differentiation and normally disappears in mature plasma cells. CD19 is closely associated with the B-cell antigen receptor and functions to send a signal when the cell engages antigen. CD20 and CD19 are expressed at very high levels on lymphoma cells. Approximately 90% of low-grade lymphomas express CD20 while CD 19 is nearly ubiquitously expressed from all B-cells excluding bone marrow progenitors and plasma cells. Thus, CD 19 is the preferred target because of its near universal expression. Unfortunately, monoclonal antibodies directed towards it appear to be less efficient than those directed to CD20 (Hooijberg et al. (1995) Cancer Research 55:840-846). Additionally, the high-level of expression by normal B-cells insures that profound immune deficiency will result when CD19 is used for the target molecule.

Thus, CD20 has become the premiere target for monoclonal therapy directed at B-cell antigens. In vitro work has demonstrated that monoclonal antibodies directed to CD20 result in cell death by apoptosis (Shan et al. (1998) Blood 91:1644-1652). Other studies report that B-cell death is primarily mediated by antibody-dependent cytotoxicity (ADCC). ADCC is a cellular mechanism that depends on specific effector cells carrying receptors for the monoclonal antibody bound to its target. These are in general receptors that are present on NK cells, neutrophils, and cells with monocyte/macrophage lineage. The NK cells appear to be the relevant mediators of this phenomenon, and antibodies to CD20 mediate their cytotoxicity primarily through ADCC.

Because of the possible immunological basis of the anti-tumor activity of anti-CD20 antibodies, combinations with promoters of NK cell function have been examined. Cytokines such as IL-12, IL-15, TNF-alpha, TNF-beta, gamma-IFN, and IL-2 have been tested for potentiation of ADCC. All appear to be active in potentiating ADCC, although each agent is associated with its own specific toxicities.

The most compelling model is a nude mouse implanted with Daudi cells. Daudi cells are cells from a cell line derived from a patient with Burkitt's lymphoma, a B-cell tumor that expresses CD20. In this model, IL-2 was tested in combination with unconjugated anti-CD20 antibody both as a prophylaxis and after tumors had been established (Hooijberg et al. (1995) Cancer Research 55:2627-2634). The Hooijberg study showed that IL-2, in combination with unconjugated anti-CD20 antibody, is able to eliminate tumors completely in some animals. The combination was highly effective at affecting complete regression of tumors. Other cytokine combinations and the use of cytokines alone were much less effective in eliminating tumors. Hooijberg et al. also examined the combination in preventing tumor outgrowth and found that IL-2 and anti-CD20 were highly effective in preventing tumor growth.

Thus, this model supports the notion that IL-2 in combination with anti-CD20 is a potent mediator of B-cell tumor regression in prevention of tumor outgrowth. However, the model's assumptions need to be carefully considered. First and foremost is the dose and schedule of administered IL-2 and antibody. The IL-2 was given weekly and in a subcutaneous dose of 200,000 units/mouse. The equivalent dose in humans could be as high as 6×10⁸ IU, which is a large, essentially unwieldy dose that is greater than high-dose bolus used in treatment of renal cell carcinoma or metastatic melanoma.

Rituximab (IDEC-C2B8; IDEC Pharmaceuticals Corp., San Diego, Calif.) is a chimeric anti-CD20 monoclonal antibody containing human IgG1 and kappa constant regions with murine variable regions isolated from a murine anti-CD20 monoclonal antibody, IDEC-2B8 (Reff et al. (1994) Blood 83:435-445). The anti-lymphoma effects of Rituximab are in part due to complement antibody-dependent cell mediated cytotoxicity, inhibition of cell proliferation, and induction of apoptosis. In early studies, Rituximab induced a rapid depletion of CD20⁺ normal B-cells and lymphoma cells (Reff et al. (1994) Blood 83:435-445). Phase I trials of single doses up to 500 mg/m² and of 4 weekly doses of 375 mg/m² demonstrated clinical responses with no dose-limiting toxicity in low-grade or follicular lymphoma patients (Maloney et al. (1994) Blood 84:2457-2466. In a phase II trial, 4 weekly infusions of 375 mg/m² induced responses in 17 of 34 evaluable low-grade or follicular lymphoma patients, with a median time to progression of 10.2 months (Maloney et al. (1997) Blood 90:2188-2195). Side effects were associated with the first Rituximab infusion and usually were mild to moderate. In a recently reported large pivotal phase II study, in 166 patients with low-grade or follicular lymphoma, objective response was reported for 76 (50%) of 151 evaluable patients and side effects were identical to those previously described (McLaughlin et al. (1998) J. Clin. Oncol. 16:2825-2833). Previous experience with Rituximab in patients with large B-cell lymphoma is very limited, with fewer than 12 patients having been included in the early phase I and phase II studies. Recent studies indicate, however, that Rituximab has significant activity in diffuse large B-cell lymphoma and mantle cell lymphoma patients and should be tested in combination with chemotherapy in such patients (Coiffier et al (1998) Blood 92:1927-1932).

However, the reality of all current antineoplastic therapies includes tumor escape, wherein clonal tumor cells develop a mechanism by which they can resist specific therapies. In a recent study it was shown that therapy of B-cell lymphoma with anti-CD20 antibodies can result in loss of the CD20 antigen expression (Davis et al (1999) Clin. Cancer Res. 5:611-615). After two courses of therapy with Rituximab, the patient developed a transformed lymphoma that no longer expressed CD20. This indicates that patients undergoing this therapy should be evaluated for CD20 expression before repeated courses of anti-CD20 therapy.

Thus, although IL-2 and Rituximab have provided a means for partial treatment of lymphoma, new therapies are needed that will provide prolonged treatment for this cancer.

### SUMMARY OF THE INVENTION

Methods for providing treatment to a mammal with lymphoma using a combination of interleukin-2 (IL-2) or variant thereof and at least one anti-CD20 antibody or fragment thereof are provided. The combination of IL-2 (or variant thereof) and at least one anti-CD20 antibody (or fragment thereof) promotes a positive therapeutic response. The methods comprise concurrent therapy with IL-2 (or variant thereof) and at least one anti-CD20 antibody (or fragment thereof). These anti-tumor agents are administered as two separate pharmaceutical compositions, one containing IL-2 (or variant thereof), the other containing at least one anti-CD20 antibody (or fragment thereof), according to a dosing regimen. Administering of these two agents together potentiates the effectiveness of either agent alone, resulting in a positive therapeutic response that is improved with respect to that observed with either agent alone. In addition, the anti-tumor effects of these agents can be achieved using lower dosages of IL-2, thereby lessening the toxicity of prolonged IL-2 administration and the potential for tumor escape.

### DETAILED DESCRIPTION OF THE INVENTION

The present invention relates to methods of treating a mammal with lymphoma, more particularly non-Hodgkin's B-cell lymphoma. The methods comprise concurrent therapy with interleukin-2 (IL-2) or variant thereof and at least one anti-CD20 antibody or fragment thereof. These two agents exhibit anti-tumor activity and hence are referred to as anti-tumor agents. By “anti-tumor activity” is intended a reduction in the rate of cell proliferation, and hence a decline in growth rate of an existing tumor or in a tumor that arises during therapy, and/or destruction of existing neoplastic (tumor) cells or newly formed neoplastic cells, and hence a decrease in the overall size of a tumor during therapy. Therapy with a combination of IL-2 (or variant thereof) and at least one anti-CD20 antibody (or fragment thereof) causes a physiological response that is beneficial with respect to treatment of non-Hodgkin's lymphoma, more particularly non-Hodgkin's B-cell lymphoma, in a mammal.

By “non-Hodgkin's B-cell lymphoma” is intended any of the non-Hodgkin's based lymphomas related to abnormal, uncontrollable B-cell proliferation. For purposes of the present invention, such lymphomas will be referred to according to the Working Formulation classification scheme, that is those B-cell lymphomas categorized as low grade, intermediate grade, and high grade (see “The Non-Hodgkin's Lymphoma Pathologic Classification Project,” Cancer 49(1982): 2112-2135). Thus, low-grade B-cell lymphomas include small lymphocytic, follicular small-cleaved cell, and follicular mixed small-cleaved and large cell lymphomas; intermediate-grade lymphomas include follicular large cell, diffuse small cleaved cell, diffuse mixed small and large cell, and diffuse large cell lymphomas; and high-grade lymphomas include large cell immunoblastic, lymphoblastic, and small non-cleaved cell lymphomas of the Burkitt's and non-Burkitt's type.

It is recognized that the methods of the invention are useful in the therapeutic treatment of B-cell lymphomas that are classified according to the Revised European and American Lymphoma Classification (REAL) system. Such B-cell lymphomas include, but are not limited to, lymphomas classified as precursor B-cell neoplasms, such as B-lymphoblastic leukemia/lymphoma; peripheral B-cell neoplasms, including B-cell chronic lymphocytic leukemia/small lymphocytic lymphoma, lymphoplasmacytoid lymphoma/immunocytoma, mantle cell lymphoma (MCL), follicle center lymphoma (follicular) (including diffuse small cell, diffuse mixed small and large cell, and diffuse large cell lymphomas), marginal zone B-cell lymphoma (including extranodal, nodal, and splenic types), hairy cell leukemia, plasmacytomal myeloma, diffuse large cell B-cell lymphoma of the subtype primary mediastinal (thymic), Burkitt's lymphoma, and Burkitt's like high grade B-cell lymphoma; and unclassifiable low-grade or high-grade B-cell lymphomas.

More particularly, the therapeutic methods of the invention are directed to treatment of any non-Hodgkin's B-cell lymphoma whose abnormal B-cell type expresses the CD20 surface antigen. By “CD20 surface antigen” is intended a 33-37 kDa integral membrane phosphoprotein that is expressed during early pre-B cell development but which is lost at the plasma cell stage. This surface antigen, also known as Bp35, may regulate a step in the activation process that is required for cell cycle initiation and differentiation. Although CD20 is expressed on normal B cells, this surface antigen is usually expressed at very high levels on neoplastic B cells. More than 90% of B-cell lymphomas and chronic lymphocytic leukemias, and about 50% of pre-B-cell acute lymphoblastic leukemias express this surface antigen.

It is recognized that concurrent therapy with IL-2 or variant thereof and an anti-CD20 antibody or fragment thereof may be useful in the treatment of any type of cancer whose unabated proliferating cells express the CD20 surface antigen. Thus, for example, where a cancer is associated with aberrant T-cell proliferation, and the aberrant T-cell population expresses the CD20 surface antigen, concurrent therapy in accordance with the methods of the invention would provide a positive therapeutic response with respect to treatment of that cancer. A human T-cell population expressing the CD20 surface antigen, though in reduced amounts relative to B-cells, has been identified (see Hultin et al. (1993) Cytometry 14:196-204).

While the methods of the invention are directed to treatment of an existing non-Hodgkin's B-cell lymphoma, it is recognized that the methods may be useful in preventing further tumor outgrowths arising during therapy. The methods of the invention are particularly useful in the treatment of subjects having low-grade B-cell lymphomas, particularly those subjects having relapses following standard chemotherapy. Low-grade B-cell lymphomas are more indolent than the intermediate- and high-grade B-cell lymphomas and are characterized by a relapsing/remitting course. Thus, treatment of these lymphomas is improved using the methods of the invention, as relapse episodes are reduced in number and severity.

The methods of the present invention may be used with any mammal. Exemplary mammals include, but are not limited to, cats, dogs, horses, cows, sheep, pigs, and more preferably humans.

In accordance with the methods of the present invention, IL-2 (or variant thereof) and at least one anti-CD20 antibody (or fragment thereof) as defined elsewhere below are used in combination to promote a positive therapeutic response with respect to non-Hodgkin's B-cell lymphoma. By “positive therapeutic response” is intended an improvement in the disease in association with the anti-tumor activity of these agents, and/or an improvement in the symptoms associated with the disease. Thus, for example, an improvement in the disease may be characterized as a complete response. By “complete response” is intended an absence of clinically detectable disease with normalization of any previously abnormal radiographic studies, bone marrow, and cerebrospinal fluid (CSF). Such a response must persist for at least one month following treatment according to the methods of the invention. Alternatively, an improvement in the disease may be categorized as being a partial response. By “partial response” is intended at least a 50% decrease in all measurable tumor burden (i.e., the number of tumor cells present in the subject) in the absence of new lesions and persisting for at least one month. Such a response is applicable to measurable tumors only. In addition to these positive therapeutic responses, the subject undergoing concurrent therapy with these two anti-tumor agents may experience the beneficial effect of an improvement in the symptoms associated with the disease. Thus the subject may experience a decrease in the so-called B symptoms, i.e., night sweats, fever, weight loss, and/or urticaria.

Promotion of a positive therapeutic response with respect to a non-Hodgkin's lymphoma in a mammal is achieved via concurrent therapy with both IL-2 (or variant thereof) and at least one anti-CD20 antibody (or fragment thereof). By “concurrent therapy” is intended presentation of IL-2 (or variant thereof) and at least one anti-CD20 antibody (or fragment thereof) to a mammal such that the therapeutic effect of the combination of both substances is caused in the mammal undergoing therapy. Concurrent therapy may be achieved by administering at least one therapeutically effective dose of a pharmaceutical composition comprising IL-2 (or variant thereof) and at least one therapeutically effective dose of a pharmaceutical composition comprising at least one anti-CD20 antibody (or fragment thereof) according to a particular dosing regimen. By “therapeutically effective dose or amount” is intended an amount of the anti-tumor agent that, when administered with a therapeutically effective dose or amount of the other anti-tumor agent, brings about a positive therapeutic response with respect to treatment of non-Hodgkin's lymphoma. Administration of the separate pharmaceutical compositions can be at the same time or at different times, so long as the therapeutic effect of the combination of both substances is caused in the mammal undergoing therapy.

The separate pharmaceutical compositions comprising these anti-tumor agents as therapeutically active components may be administered using any acceptable method known in the art. Preferably the pharmaceutical composition comprising IL-2 or variant thereof is administered by any form of injection, more preferably intravenous (IV) or subcutaneous (SC) injection, most preferably SC injection, and preferably the pharmaceutical composition comprising the monoclonal antibody is administered intravenously, preferably by infusion over a period of about 1 to about 10 hours, more preferably over about 2 to about 8 hours, even more preferably over about 3 to about 7 hours, still more preferably over about 4 to about 6 hours, most preferably over about 6 hours, depending upon the anti-CD20 antibody being administered.

Concurrent therapy with an effective amount of the combination of IL-2 (or variant thereof) and at least one anti-CD20 antibody (or fragment thereof) promotes a positive therapeutic response with respect to non-Hodgkin's B-cell lymphoma. The respective amounts of IL-2 (or variant thereof) and at least one anti-CD20 antibody (or fragment thereof) that in combination promote the positive therapeutic response are a function of one another. Thus, the amount (or dose) of IL-2 (or variant thereof) to be used during concurrent therapy is a function of the amount (or dose) of at least one anti-CD20 antibody (or fragment thereof) being used in combination with a given dose of IL-2 (or variant thereof). Likewise, the amount of at least one anti-CD20 antibody (or fragment thereof) to be used during concurrent therapy is a function of the amount of IL-2 (or variant thereof) being used in combination with a given dose of at least one anti-CD20 antibody (or fragment thereof). Concurrent therapy with both of these anti-tumor agents potentiates the anti-tumor activity of each of these agents, thereby providing a positive therapeutic response that is improved with respect to that observed with administration of IL-2 (or variant thereof) alone or at least one anti-CD20 antibody (or fragment thereof) alone. Improvement of the positive therapeutic response may be additive in nature or synergistic in nature. Where synergistic, concurrent therapy with IL-2 (or variant thereof) and at least one anti-CD20 antibody (or fragment thereof) results in a positive therapeutic response that is greater than the sum of the positive therapeutic responses achieved with the separate IL-2 (or variant thereof) and anti-CD20 antibody (or fragment thereof) components.

Because the combined administration of these two anti-tumor agents potentiates the effectiveness of both of these agents, a positive therapeutic response that is similar to that achieved with a particular dose of IL-2 alone can be achieved with lower doses of this agent. Thus, a dose of IL-2 alone that is not normally therapeutically effective may be therapeutically effective when administered in combination with at least one anti-CD20 antibody in accordance with the methods of the invention. The significance of this is two-fold. First, the potential therapeutic benefits of treatment of lymphoma with IL-2 or variant thereof can be realized at IL-2 doses that minimize toxicity responses normally associated with prolonged IL-2 therapy or high-bolus IL-2 administration. Such toxicity responses include, but are not limited to, chronic fatique, nausea, hypotension, fever, chills, weight gain, pruritis or rash, dysprea, azotemia, confusion, thrombocytopenia, myocardial infarction, gastrointestinal toxicity, and vascular leak syndrome (see, for example, Allison et al. (1989) J Clin. Oncol. 7(1): 75-80; and Gisselbrecht et al. (1994) Blood 83(8): 2081-2085). Secondly, targeting of specific molecules on a tumor cell surface by monoclonal antibodies can select for clones that are not recognized by the antibody or are not affected by its binding, resulting in tumor escape, and loss of effective therapeutic treatment. Such tumor escape has been documented with repeated doses of an anti-CD20 antibody (Davis et al. (1999) Clin. Cancer Res. 5:611-615). Improved anti-tumor activity of anti-CD20 antibodies or fragment thereof administered in combination with IL-2 or variant thereof may translate into less frequent administration of monoclonal antibodies, thereby lessening the potential for tumor escape.

The amount of at least one anti-CD20 antibody or fragment thereof to be administered in combination with an amount of IL-2 (or variant thereof) and the amount of either anti-tumor agent needed to potentiate the effectiveness of the other anti-tumor agent are readily determined by one of ordinary skill in the art without undue experimentation. Factors influencing the mode of administration and the respective amount of IL-2 (or variant thereof) administered in combination with a given amount of at least one anti-CD20 antibody (or fragment thereof) include, but are not limited to, the particular lymphoma undergoing therapy, the severity of the disease, the history of the disease, and the age, height, weight, health, and physical condition of the individual undergoing therapy. Similarly, the amount of these anti-tumor agents to be administered concurrently will be dependent upon the mode of administration and whether the subject will undergo a single dose or multiple doses of each of the anti-tumor agents. Generally, a higher dosage of these agents is preferred with increasing weight of the mammal undergoing therapy.

Thus, the amount of IL-2 (or variant thereof) to be administered as a therapeutically effective dose is a function of the amount of at least one anti-CD20 antibody administered in combination with the IL-2 (or variant thereof) and vice versa. For example, in one embodiment, the therapeutically effective dose of IL-2 (or variant thereof) to be administered concurrently with at least one anti-CD20 antibody (or fragment thereof) is in the range from about 1 mIU/m² to about 14 mIU/m², preferably from about 2 mIU/m² to about 12 mIU/m², more preferably from about 3 mIU/m² to about 6 mIU/m², most preferably about 4.5 mIU/m², while the therapeutically effective dose of at least one anti-CD20 antibody is in the range from about 100 mg/m² to about 550 mg/m², preferably about 125 mg/m² to about 500 mg/m², more preferably about 225 mg/m² to about 400 mg/m², most preferably about 375 mg/m². When the amount of IL-2 (or variant thereof) is about 3 mIU/m² to about 6 mIU/m²/dose, preferably the total amount of anti-CD20 antibody or fragment thereof, which comprises at least one anti-CD20 antibody (or fragment thereof), is about 225 mg/m²/dose to about 400 mg/m²/dose. Thus, for example, the amount of IL-2 or variant thereof could be about 3.0, 3.5, 4.0, 4.5, 5.0, 5.5, or 6.0 mIU/m²/dose and the total amount of anti-CD20 antibody could be about 225, 250, 275, 300, 325, 350, 375, or 400 mg/m²/dose. When the amount of IL-2 or variant thereof is about 4.5 mIU/m²/dose, preferably the amount of anti-CD20 antibody is about 325, 350, 375, or 400 mg/m²/dose, most preferably about 375 mg/m²/dose.

Concurrent therapy with one therapeutically effective dose of IL-2 or variant thereof and one therapeutically effective dose of at least one anti-CD20 antibody or fragment thereof is beneficial with respect to treatment/management of non-Hodgkin's B-cell lymphoma. Generally, the initial anti-tumor agent to be administered is anti-CD20 antibody or fragment thereof, while the IL-2 or variant thereof is administered subsequently. Depending upon the severity of the disease, the patient's health, and prior history of the patient's disease, concurrent therapy with multiple doses of IL-2 or variant thereof and at least one anti-CD20 antibody or variant thereof is preferred. Thus, for example, in one embodiment, the preferred dosing regimen includes a first administration of a therapeutically effective dose of at least one anti-CD20 antibody or fragment thereof on day 1 of a treatment period, followed by a first administration of a therapeutically effective dose of the IL-2 or variant thereof within 7 days of the first administration of the anti-CD20 antibody, such as within 1, 2, 3, 4, 5, 6, or 7 days, preferably within about 2 to about 4 days, more preferably within about 3 days. In another embodiment, the preferred dosing regimen includes a first administration of a therapeutically effective dose of at least one anti-CD20 antibody or fragment thereof on days 1, 8, 15, and 22 of a treatment period, with daily administration of a therapeutically effective dose of IL-2 or variant thereof beginning on day 3, 4, 5, 6, 7, 8, 9, or 10, preferably on day 3, 5, 7, or 8, most preferably on day 8 of the same treatment period and running daily through day 22, 23, 24, 25, 26, 27, 28,29, 30, 31, 32, 33, 34, 35, or 36, preferably through day 23, more preferably through day 25, even more preferably through day 27, most preferably through day 29 of the same treatment period. In yet another embodiment, the preferred dosing regimen includes a first administration of a therapeutically effective dose of at least one anti-CD20 antibody or fragment thereof on days 1, 8, 15, and 22 of a treatment period, with a therapeutically effective dose of IL-2 or variant thereof administered beginning on day 3, 4, 5, 6, 7, 8, 9, or 10, preferably on day 3, 5, 7, or 8, most preferably beginning on day 8 of the same treatment period, with subsequent administration of therapeutically effective doses of IL-2 occurring three times per week thereafter for an additional consecutive 2, 3, or 4 weeks of the same treatment period, more preferably 2 or 3 weeks, most preferably an additional consecutive 3 weeks of the same treatment period. Thus, for example, therapeutically effective doses of anti-CD20 antibody or fragment thereof are administered on days 1, 8, 15, and 22 of a treatment period, while therapeutically effective doses of IL-2 or variant thereof are administered on days 8, 10, 12, 15, 17, 19, 22, 24, 26, 29, 31, 33, and 36 of the same treatment period, more preferably on days 8, 10, 12, 15, 17, 19, 22, 24, 26, and 29 of the same treatment period.

Where a subject undergoing therapy in accordance with the previously mentioned dosing regimens exhibits a partial response, or a relapse following a prolonged period of remission, subsequent courses of concurrent therapy may be needed to achieve complete remission of the disease. Thus, subsequent to a period of time off from a first treatment period, which may have comprised a single dosing regimen or a multiple dosing regimen, a subject may receive one or more additional treatment periods comprising either single or multiple dosing regimens. Such a period of time off between treatment periods is referred to herein as a time period of discontinuance. It is recognized that the length of the time period of discontinuance is dependent upon the degree of tumor response (i.e., complete versus partial) achieved with any prior treatment periods of concurrent therapy with these two anti-tumor agents.

The term “IL-2” as used herein refers to a lymphokine that is produced by normal peripheral blood lymphocytes and is present in the body at low concentrations. IL-2 was first described by Morgan et al. (1976) Science 193:1007-1008 and originally called T cell growth factor because of its ability to induce proliferation of stimulated T lymphocytes. It is a protein with a reported molecular weight in the range of 13,000 to 17,000 (Gillis and Watson (1980) J Exp. Med. 159:1709) and has an isoelectric point in the range of 6-8.5.

The IL-2 present in the pharmaceutical compositions described herein for use in the methods of the invention may be native or obtained by recombinant techniques, and may be from any source, including mammalian sources such as, e.g., mouse, rat, rabbit, primate, pig, and human. Preferably such polypeptides are derived from a human source, and more preferably are recombinant, human proteins from microbial hosts.

The pharmaceutical compositions useful in the methods of the invention may comprise biologically active variants of IL-2. Such variants should retain the desired biological activity of the native polypeptide such that the pharmaceutical composition comprising the variant polypeptide has the same therapeutic effect as the pharmaceutical composition comprising the native polypeptide when administered to a subject. That is, the variant polypeptide will serve as a therapeutically active component in the pharmaceutical composition in a manner similar to that observed for the native polypeptide. Methods are available in the art for determining whether a variant polypeptide retains the desired biological activity, and hence serves as a therapeutically active component in the pharmaceutical composition. Biological activity can be measured using assays specifically designed for measuring activity of the native polypeptide or protein, including assays described in the present invention. Additionally, antibodies raised against a biologically active native polypeptide can be tested for their ability to bind to the variant polypeptide, where effective binding is indicative of a polypeptide having a conformation similar to that of the native polypeptide.

Suitable biologically active variants of native or naturally occurring IL-2 can be fragments, analogues, and derivatives of that polypeptide. By “fragment” is intended a polypeptide consisting of only a part of the intact polypeptide sequence and structure, and can be a C-terminal deletion or N-terminal deletion of the native polypeptide. By “analogue” is intended an analogue of either the native polypeptide or of a fragment of the native polypeptide, where the analogue comprises a native polypeptide sequence and structure having one or more amino acid substitutions, insertions, or deletions. “Muteins”, such as those described herein, and peptides having one or more peptoids (peptide mimics) are also encompassed by the term analogue (see International Publication No. WO 91/04282). By “derivative” is intended any suitable modification of the native polypeptide of interest, of a fragment of the native polypeptide, or of their respective analogues, such as glycosylation, phosphorylation, polymer conjugation (such as with polyethylene glycol), or other addition of foreign moieties, so long as the desired biological activity of the native polypeptide is retained. Methods for making polypeptide fragments, analogues, and derivatives are generally available in the art.

For example, amino acid sequence variants of the polypeptide can be prepared by mutations in the cloned DNA sequence encoding the native polypeptide of interest. Methods for mutagenesis and nucleotide sequence alterations are well known in the art. See, for example, Walker and Gaastra, eds. (1983) Techniques in Molecular Biology (MacMillan Publishing Company, New York); Kunkel (1985) Proc. Natl. Acad. Sci. USA 82:488-492; Kunkel et al. (1987) Methods Enzymol. 154:367-382; Sambrook et al. (1989) Molecular Cloning: A Laboratory Manual (Cold Spring Harbor, N.Y.); U.S. Pat. No. 4,873,192; and the references cited therein; herein incorporated by reference. Guidance as to appropriate amino acid substitutions that do not affect biological activity of the polypeptide of interest may be found in the model of Dayhoff et al. (1978) in Atlas of Protein Sequence and Structure (Natl. Biomed. Res. Found., Washington, D.C.), herein incorporated by reference. Conservative substitutions, such as exchanging one amino acid with another having similar properties, may be preferred. Examples of conservative substitutions include, but are not limited to, Gly [symbol] Ala, Val [symbol] Ile [symbol] Leu, Asp [symbol] Glu, Lys [symbol] Arg, Asn [symbol] Gln, and Phe [symbol] Trp [symbol] Tyr.

In constructing variants of the IL-2 polypeptide of interest, modifications are made such that variants continue to possess the desired activity. Obviously, any mutations made in the DNA encoding the variant polypeptide must not place the sequence out of reading frame and preferably will not create complementary regions that could produce secondary MRNA structure. See EP Patent Application Publication No. 75,444.

Biologically active variants of IL-2 will generally have at least 70%, preferably at least 80%, more preferably about 90% to 95% or more, and most preferably about 98% or more amino acid sequence identity to the amino acid sequence of the reference polypeptide molecule, which serves as the basis for comparison. Thus, where the IL-2 reference molecule is human IL-2, a biologically active variant thereof will have at least 70%, preferably at least 80%, more preferably about 90% to 95% or more, and most preferably about 98% or more sequence identity to the amino acid sequence for human IL-2. A biologically active variant of a native polypeptide of interest may differ from the native polypeptide by as few as 1-15 amino acids, as few as 1-10, such as 6-10, as few as 5, as few as 4, 3, 2, or even 1 amino acid residue. By “sequence identity” is intended the same amino acid residues are found within the variant polypeptide and the polypeptide molecule that serves as a reference when a specified, contiguous segment of the amino acid sequence of the variants is aligned and compared to the amino acid sequence of the reference molecule. The percentage sequence identity between two amino acid sequences is calculated by determining the number of positions at which the identical amino acid residue occurs in both sequences to yield the number of matched positions, dividing the number of matched positions by the total number of positions in the segment undergoing comparison to the reference molecule, and multiplying the result by 100 to yield the percentage of sequence identity.

For purposes of optimal alignment of the two sequences, the contiguous segment of the amino acid sequence of the variants may have additional amino acid residues or deleted amino acid residues with respect to the amino acid sequence of the reference molecule. The contiguous segment used for comparison to the reference amino acid sequence will comprise at least twenty (20) contiguous amino acid residues, and may be 30, 40, 50, 100, or more residues. Corrections for increased sequence identity associated with inclusion of gaps in the variants' amino acid sequence can be made by assigning gap penalties. Methods of sequence alignment are well known in the art for both amino acid sequences and for the nucleotide sequences encoding amino acid sequences.

Thus, the determination of percent identity between any two sequences can be accomplished using a mathematical algorithm. One preferred, non-limiting example of a mathematical algorithm utilized for the comparison of sequences is the algorithm of Myers and Miller (1988) CABIOS 4:11-17. Such an algorithm is utilized in the ALIGN program (version 2.0), which is part of the GCG sequence alignment software package. A PAM120 weight residue table, a gap length penalty of 12, and a gap penalty of 4 can be used with the ALIGN program when comparing amino acid sequences. Another preferred, nonlimiting example of a mathematical algorithm for use in comparing two sequences is the algorithm of Karlin and Altschul (1990) Proc. Natl. Acad. Sci. USA 87:2264, modified as in Karlin and Altschul (1993) Proc. Natl. Acad. Sci. USA 90:5873-5877. Such an algorithm is incorporated into the NBLAST and XBLAST programs of Altschul et al. (1990) J Mol. Biol. 215:403. BLAST nucleotide searches can be performed with the NBLAST program, score=100, wordlength=12, to obtain nucleotide sequences homologous to a nucleotide sequence encoding the polypeptide of interest. BLAST protein searches can be performed with the XBLAST program, score=50, wordlength=3, to obtain amino acid sequences homologous to the polypeptide of interest. To obtain gapped alignments for comparison purposes, Gapped BLAST can be utilized as described in Altschul et al. (1997) Nucleic Acids Res. 25:3389. Alternatively, PSI-Blast can be used to perform an iterated search that detects distant relationships between molecules. See Altschul et al. (1997) supra. When utilizing BLAST, Gapped BLAST, and PSI-Blast programs, the default parameters of the respective programs (e.g., XBLAST and NBLAST) can be used. See http://www.ncbi.nlm.nih.gov. Also see the ALIGN program (Dayhoff (1978) in Atlas of Protein Sequence and Structure 5: Suppl. 3 (National Biomedical Research Foundation, Washington, D.C.) and programs in the Wisconsin Sequence Analysis Package, Version 8 (available from Genetics Computer Group, Madison, Wis.), for example, the GAP program, where default parameters of the programs are utilized.

When considering percentage of amino acid sequence identity, some amino acid residue positions may differ as a result of conservative amino acid substitutions, which do not affect properties of protein function. In these instances, percent sequence identity may be adjusted upwards to account for the similarity in conservatively substituted amino acids. Such adjustments are well known in the art. See, for example, Myers and Miller (1988) Computer Applic. Biol. Sci. 4:11-17.

The precise chemical structure of a polypeptide having IL-2 activity depends on a number of factors. As ionizable amino and carboxyl groups are present in the molecule, a particular polypeptide may be obtained as an acidic or basic salt, or in neutral form. All such preparations that retain their biological activity when placed in suitable environmental conditions are included in the definition of polypeptides having IL-2 activity as used herein. Further, the primary amino acid sequence of the polypeptide may be augmented by derivatization using sugar moieties (glycosylation) or by other supplementary molecules such as lipids, phosphate, acetyl groups and the like. It may also be augmented by conjugation with saccharides. Certain aspects of such augmentation are accomplished through post-translational processing systems of the producing host; other such modifications may be introduced in vitro. In any event, such modifications are included in the definition of an IL-2 polypeptide used herein so long as the IL-2 activity of the polypeptide is not destroyed. It is expected that such modifications may quantitatively or qualitatively affect the activity, either by enhancing or diminishing the activity of the polypeptide, in the various assays. Further, individual amino acid residues in the chain may be modified by oxidation, reduction, or other derivatization, and the polypeptide may be cleaved to obtain fragments that retain activity. Such alterations that do not destroy activity do not remove the polypeptide sequence from the definition of IL-2 polypeptides of interest as used herein.

The art provides substantial guidance regarding the preparation and use of polypeptide variants. In preparing the IL-2 variants, one of skill in the art can readily determine which modifications to the native protein nucleotide or amino acid sequence will result in a variant that is suitable for use as a therapeutically active component of a pharmaceutical composition used in the methods of the present invention.

The IL-2 or variants thereof for use in the methods of the present invention may be from any source, but preferably is recombinant IL-2. By “recombinant IL-2” is intended interleukin-2 that has comparable biological activity to native-sequence IL-2 and that has been prepared by recombinant DNA techniques as described, for example, by Taniguchi et al. (1983) Nature 302:305-310 and Devos (1983) Nucleic Acids Research 11:4307-4323 or mutationally altered IL-2 as described by Wang et al. (1984) Science 224:1431-1433. In general, the gene coding for IL-2 is cloned and then expressed in transformed organisms, preferably a microorganism, and most preferably E. coli, as described herein. The host organism expresses the foreign gene to produce IL-2 under expression conditions. Synthetic recombinant IL-2 can also be made in eukaryotes, such as yeast or human cells. Processes for growing, harvesting, disrupting, or extracting the IL-2 from cells are substantially described in, for example, U.S. Pat. Nos. 4,604,377; 4,738,927; 4,656,132; 4,569,790; 4,748,234; 4,530,787; 4,572,798; 4,748,234; and 4,931,543, herein incorporated by reference in their entireties.

For examples of variant IL-2 proteins, see European Patent Application No. 136,489; European Patent Application No. 83101035.0 filed Feb. 3, 1983 (published Oct. 19, 1983 under Publication No. 91539); European Patent Application No. 82307036.2, filed Dec. 22, 1982 (published Sep. 14, 1983 under No. 88195); the recombinant IL-2 muteins described in European Patent Application No. 83306221.9, filed Oct. 13, 1983 (published May 30, 1984 under No. 109748), which is the equivalent to Belgian Patent No. 893,016, commonly owned U.S. Pat. No. 4,518,584; the muteins described in U.S. Pat. No. 4,752,585 and WO 99/60128; and the IL-2 mutein (des-alanyl-1, serine-125 human interleukin-2) used in the examples herein and described in U.S. Pat. No. 4,931,543, as well as the other IL-2 muteins described in this U.S. patent; all of which are herein incorporated by reference. Additionally, IL-2 can be modified with polyethylene glycol to provide enhanced solubility and an altered pharmokinetic profile (see U.S. Pat. No. 4,766,106, hereby incorporated by reference in its entirety).

Any pharmaceutical composition comprising IL-2 as the therapeutically active component can be used in the methods of the invention. Such pharmaceutical compositions are known in the art and include, but are not limited to, those disclosed in U.S. Pat. Nos. 4,745,180; 4,766,106; 4,816,440; 4,894,226; 4,931,544; and 5,078,997; herein incorporated by reference. Thus liquid, lyophilized, or spray-dried compositions comprising IL-2 or variants thereof that are known in the art may be prepared as an aqueous or nonaqueous solution or suspension for subsequent administration to a subject in accordance with the methods of the invention. Each of these compositions will comprise IL-2 or variants thereof as a therapeutically or prophylactically active component. By “therapeutically or prophylactically active component” is intended the IL-2 or variants thereof is specifically incorporated into the composition to bring about a desired therapeutic or prophylactic response with regard to treatment, prevention, or diagnosis of a disease or condition within a subject when the pharmaceutical composition is administered to that subject. Preferably the pharmaceutical compositions comprise appropriate stabilizing agents, bulking agents, or both to minimize problems associated with loss of protein stability and biological activity during preparation and storage.

In preferred embodiments of the invention, the IL-2 containing pharmaceutical compositions useful in the methods of the invention are compositions comprising stabilized monomeric IL-2 or variants thereof, compositions comprising multimeric IL-2 or variants thereof, and compositions comprising stabilized lyophilized or spray-dried IL-2 or variants thereof.

Pharmaceutical compositions comprising stabilized monomeric IL-2 or variants thereof are disclosed in the copending application entitled “Stabilized Liquid Polypeptide-Containing Pharmaceutical Compositions,” filed October 3, 2000, and assigned U.S. application Ser. No. 09/677,643, the disclosure of which is herein incorporated by reference. By “monomeric” IL-2 is intended the protein molecules are present substantially in their monomer form, not in an aggregated form, in the pharmaceutical compositions described herein. Hence covalent or hydrophobic oligomers or aggregates of IL-2 are not present. Briefly, the IL-2 or variants thereof in these liquid compositions is formulated with an amount of an amino acid base sufficient to decrease aggregate formation of IL-2 or variants thereof during storage. The amino acid base is an amino acid or a combination of amino acids, where any given amino acid is present either in its free base form or in its salt form. Preferred amino acids are selected from the group consisting of arginine, lysine, aspartic acid, and glutamic acid. These compositions further comprise a buffering agent to maintain pH of the liquid compositions within an acceptable range for stability of IL-2 or variants thereof, where the buffering agent is an acid substantially free of its salt form, an acid in its salt form, or a mixture of an acid and its salt form. Preferably the acid is selected from the group consisting of succinic acid, citric acid, phosphoric acid, and glutamic acid. Such compositions are referred to herein as stabilized monomeric IL-2 pharmaceutical compositions.

The amino acid base in these compositions serves to stabilize the IL-2 or variants thereof against aggregate formation during storage of the liquid pharmaceutical composition, while use of an acid substantially free of its salt form, an acid in its salt form, or a mixture of an acid and its salt form as the buffering agent results in a liquid composition having an osmolarity that is nearly isotonic. The liquid pharmaceutical composition may additionally incorporate other stabilizing agents, more particularly methionine, a nonionic surfactant such as polysorbate 80, and EDTA, to further increase stability of the polypeptide. Such liquid pharmaceutical compositions are said to be stabilized, as addition of amino acid base in combination with an acid substantially free of its salt form, an acid in its salt form, or a mixture of an acid and its salt form, results in the compositions having increased storage stability relative to liquid pharmaceutical compositions formulated in the absence of the combination of these two components.

These liquid pharmaceutical compositions comprising stabilized monomeric IL-2 or variants thereof may either be used in an aqueous liquid form, or stored for later use in a frozen state, or in a dried form for later reconstitution into a liquid form or other form suitable for administration to a subject in accordance with the methods of present invention. By “dried form” is intended the liquid pharmaceutical composition or formulation is dried either by freeze drying (i.e., lyophilization; see, for example, Williams and Polli (1984) J Parenteral Sci. Technol. 38:48-59), spray drying (see Masters (1991) in Spray-Drying Handbook (5th ed; Longman Scientific and Technical, Essez, U.K.), pp. 491-676; Broadhead et al. (1992) Drug Devel. Ind. Pharm. 18:1169-1206; and Mumenthaler et al. (1994) Pharm. Res. 11:12-20), or air drying (Carpenter and Crowe (1988) Cryobiology 25:459-470; and Roser (1991) Biopharm. 4:47-53).

Examples of pharmaceutical compositions comprising multimeric IL-2 or variants thereof are disclosed in commonly owned U.S. Pat. No. 4,604,377, the disclosure of which is herein incorporated by reference. By “multimeric” is intended the protein molecules are present in the pharmaceutical composition in a microaggregated form having an average molecular association of 10-50 molecules. These multimers are present as loosely bound, physically-associated IL-2 molecules. A lyophilized form of these compositions is available commercially under the tradename Proleukin (Chiron Corporation). The lyophilized formulations disclosed in this reference comprise selectively oxidized, microbially produced recombinant IL-2 in which the recombinant IL-2 is admixed with a water soluble carrier such as mannitol that provides bulk, and a sufficient amount of sodium dodecyl sulfate to ensure the solubility of the recombinant IL-2 in water. These compositions are suitable for reconstitution in aqueous injections for parenteral administration and are stable and well tolerated in human patients. When reconstituted, the IL-2 or variants thereof retains its multimeric state. Such lyophilized or liquid compositions comprising multimeric IL-2 or variants thereof are encompassed by the methods of the present invention. Such compositions are referred to herein as multimeric IL-2 pharmaceutical compositions.

The methods of the present invention may also use stabilized lyophilized or spray-dried pharmaceutical compositions comprising IL-2 or variants thereof, which may be reconstituted into a liquid or other suitable form for administration in accordance with methods of the invention. Such pharmaceutical compositions are disclosed in the copending application entitled “Methods for Pulmonary Delivery of Interleukin-2,” U.S. application Ser. No. 09/724,810, filed Nov. 28, 2000, herein incorporated by reference. These compositions may further comprise at least one bulking agent, at least one agent in an amount sufficient to stabilize the protein during the drying process, or both. By “stabilized” is intended the IL-2 protein or variants thereof retains its monomeric or multimeric form as well as its other key properties of quality, purity, and potency following lyophilization or spray-drying to obtain the solid or dry powder form of the composition. In these compositions, preferred carrier materials for use as a bulking agent include glycine, mannitol, alanine, valine, or any combination thereof, most preferably glycine. The bulking agent is present in the formulation in the range of 0% to about 10% (w/v), depending upon the agent used. Preferred carrier materials for use as a stabilizing agent include any sugar or sugar alcohol or any amino acid. Preferred sugars include sucrose, trehalose, raffinose, stachyose, sorbitol, glucose, lactose, dextrose or any combination thereof, preferably sucrose. When the stabilizing agent is a sugar, it is present in the range of about 0% to about 9.0% (w/v), preferably about 0.5% to about 5.0%, more preferably about 1.0% to about 3.0%, most preferably about 1.0%. When the stabilizing agent is an amino acid, it is present in the range of about 0% to about 1.0% (w/v), preferably about 0.3% to about 0.7%, most preferably about 0.5%. These stabilized lyophilized or spray-dried compositions may optionally comprise methionine, ethylenediaminetetracetic acid (EDTA) or one of its salts such as disodium EDTA or other chelating agent, which protect the IL-2 or variants thereof against methionine oxidation. Use of these agents in this manner is described in copending U.S. application Ser. No. 09/677,643, herein incorporated by reference. The stabilized lyophilized or spray-dried compositions may be formulated using a buffering agent, which maintains the pH of the pharmaceutical composition within an acceptable range, preferably between about pH 4.0 to about pH 8.5, when in a liquid phase, such as during the formulation process or following reconstitution of the dried form of the composition. Buffers are chosen such that they are compatible with the drying process and do not affect the quality, purity, potency, and stability of the protein during processing and upon storage.

The previously described stabilized monomeric, multimeric, and stabilized lyophilized or spray-dried IL-2 pharmaceutical compositions represent suitable compositions for use in the methods of the invention. However, any pharmaceutical composition comprising IL-2 or variant thereof as a therapeutically active component is encompassed by the methods of the invention.

As used herein, the term “anti-CD20 antibody” encompasses any antibody that specifically recognizes the CD20 B-cell surface antigen. Preferably the antibody is monoclonal in nature. By “monoclonal antibody” is intended an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site, i.e., the CD20 B-cell surface antigen in the present invention. Furthermore, in contrast to conventional (polyclonal) antibody preparations that typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al. (1975) Nature 256:495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352:624-628 and Marks et al. (1991) J Mol. Biol. 222:581-597, for example.

Anti-CD20 antibodies of murine origin are suitable for use in the methods of the present invention. Examples of such murine anti-CD20 antibodies include, but are not limited to, the BI antibody (described in U.S. Pat. No. 6,015,542); the IF5 antibody (see Press et al. (1989) J Clin. Oncol. 7:1027); NKI-B20 and BCA-B20 anti-CD20 antibodies (described in Hooijberg et al. (1995) Cancer Research 55:840-846); and IDEC-2B8 (available commercially from IDEC Pharmaceuticals Corp., San Diego, Calif.); the 2H7 antibody (described in Clark et al. (1985) Proc. Natl. Acad. Sci. USA 82:1766-1770; and others described in Clark et al. (1985) supra and Stashenko et al. (1980) J Immunol. 125:1678-1685; herein incorporated by reference.

The term “anti-CD20 antibody” as used herein encompasses chimeric anti-CD20 antibodies. By “chimeric antibodies” is intended antibodies that are most preferably derived using recombinant deoxyribonucleic acid techniques and which comprise both human (including immunologically “related” species, e.g., chimpanzee) and non-human components. Thus, the constant region of the chimeric antibody is most preferably substantially identical to the constant region of a natural human antibody; the variable region of the chimeric antibody is most preferably derived from a non-human source and has the desired antigenic specificity to the CD20 cell surface antigen. The non-human source can be any vertebrate source that can be used to generate antibodies to a human CD20 cell surface antigen or material comprising a human CD20 cell surface antigen. Such non-human sources include, but are not limited to, rodents (e.g., rabbit, rat, mouse, etc.; see, for example, U.S. Pat. No. 4,816,567, herein incorporated by reference) and non-human primates (e.g., Old World Monkey, Ape, etc.; see, for example, U.S. Pat. Nos. 5,750,105 and 5,756,096; herein incorporated by reference). Most preferably, the non-human component (variable region) is derived from a murine source. As used herein, the phrase “immunologically active” when used in reference to chimeric anti-CD20 antibodies means a chimeric antibody that binds human C1q, mediates complement dependent lysis (“CDC”) of human B lymphoid cell lines, and lyses human target cells through antibody dependent cellular cytotoxicity (“ADCC”). Examples of chimeric anti-CD20 antibodies include, but are not limited to, IDEC-C2BS, available commercially under the name Rituximab (IDEC Pharmaceuticals Corp., San Diego, Calif.) and described in U.S. Pat. Nos. 5,736,137, 5,776,456, and 5,843,439; the chimeric antibodies described in U.S. Pat. No. 5,750,105; those described in U.S. Pat. Nos. 5,500,362; 5,677,180; 5,721,108; and 5,843,685; herein incorporated by reference.

Humanized anti-CD20 antibodies are also encompassed by the term anti-CD20 antibody as used herein. By “humanized” is intended forms of anti-CD20 antibodies that contain minimal sequence derived from non-human immunoglobulin sequences. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. See, for example, U.S. Pat. Nos. 5,225,539; 5,585,089; 5,693,761; 5,693,762; 5,859,205; herein incorporated by reference. In some instances, framework residues of the human immunoglobulin are replaced by corresponding non-human residues (see, for example, U.S. Pat. Nos. 5,585,089; 5,693,761; 5,693,762). Furthermore, humanized antibodies may comprise residues that are not found in the recipient antibody or in the donor antibody. These modifications are made to further refine antibody performance (e.g., to obtain desired affinity). In general, the humanized antibody will comprise substantially all of at least one, and typically two, variable domains, in which all or substantially all of the hypervariable regions correspond to those of a non-human immunoglobulin and all or substantially all of the framework regions are those of a human immunoglobulin sequence. The humanized antibody optionally also will comprise at least a portion of an immunoglobulin constant region (Fc), typically that of a human immunoglobulin. For further details see Jones et al. (1986) Nature 331:522-525; Riechmann et al (1988) Nature 332:323-329; and Presta (1992) Curr. Op. Struct. Biol. 2:593-596; herein incorporated by reference.

Also encompassed by the term anti-CD20 antibodies are xenogeneic or modified anti-CD20 antibodies produced in a non-human mammalian host, more particularly a transgenic mouse, characterized by inactivated endogenous immunoglobulin (Ig) loci. In such transgenic animals, competent endogenous genes for the expression of light and heavy subunits of host immunoglobulins are rendered non-functional and substituted with the analogous human immunoglobulin loci. These transgenic animals produce human antibodies in the substantial absence of light or heavy host immunoglobulin subunits. See, for example, U.S. Pat. No. 5,939,598, herein incorporated by reference.

Fragments of the anti-CD20 antibodies are suitable for use in the methods of the invention so long as they retain the desired affinity of the full-length antibody. Thus, a fragment of an anti-CD20 antibody will retain the ability to bind to the CD20 B-cell surface antigen. Fragments of an antibody comprise a portion of a full-length antibody, generally the antigen binding or variable region thereof. Examples of antibody fragments include, but are not limited to, Fab, Fab′ F(ab′)₂, and Fv fragments and single-chain antibody molecules. By “single-chain Fv” or “sFv” antibody fragments is intended fragments comprising the V_{H} and V_{L} domains of an antibody, wherein these domains are present in a single polypeptide chain. See, for example, U.S. Pat. Nos. 4,946,778; 5,260,203; 5,455,030; 5,856,456; herein incorporated by reference. Generally, the Fv polypeptide further comprises a polypeptide linker between the V_{H} and V_{L} domains that enables the sFv to form the desired structure for antigen binding. For a review of sFv see Pluckthun (1994) in The Pharmacology of Monoclonal Antibodies, Vol. 113, ed. Rosenburg and Moore (Springer-Verlag, New York), pp. 269-315.

Antibodies or antibody fragments can be isolated from antibody phage libraries generated using the techniques described in McCafferty et al. (1990) Nature 348:552-554 (1990). Clackson et al. (1991) Nature 352:624-628 and Marks et al. (1991) J Mol. Biol. 222:581-597 describe the isolation of murine and human antibodies, respectively, using phage libraries. Subsequent publications describe the production of high affinity (nM range) human antibodies by chain shuffling (Marks et al. (1992) Bio/Technology 10:779-783), as well as combinatorial infection and in vivo recombination as a strategy for constructing very large phage libraries (Waterhouse et al. (1993) Nucleic. Acids Res. 21:2265-2266). Thus, these techniques are viable alternatives to traditional monoclonal antibody hybridoma techniques for isolation of monoclonal antibodies.

A humanized antibody has one or more amino acid residues introduced into it from a source that is non-human. These non-human amino acid residues are often referred to as “donor” residues, which are typically taken from a “donor” variable domain. Humanization can be essentially performed following the method of Winter and co-workers (Jones et al. (1986) Nature 321:522-525; Riechmann et al. (1988) Nature 332:323-327; Verhoeyen et al. (1988) Science 239:1534-1536), by substituting rodent CDRs or CDR sequences for the corresponding sequences of a human antibody. See, for example, U.S. Pat. Nos. 5,225,539; 5,585,089; 5,693,761; 5,693,762; 5,859,205; herein incorporated by reference. Accordingly, such “humanized” antibodies may include antibodies wherein substantially less than an intact human variable domain has been substituted by the corresponding sequence from a non-human species. In practice, humanized antibodies are typically human antibodies in which some CDR residues and possibly some framework residues are substituted by residues from analogous sites in rodent antibodies. See, for example, U.S. Pat. Nos. 5,225,539; 5,585,089; 5,693,761; 5,693,762; 5,859,205.

Various techniques have been developed for the production of antibody fragments. Traditionally, these fragments were derived via proteolytic digestion of intact antibodies (see, e.g., Morimoto et al. (1992) Journal of Biochemical and Biophysical Methods 24:107-117 (1992) and Brennan et al. (1985) Science 229:81). However, these fragments can now be produced directly by recombinant host cells. For example, the antibody fragments can be isolated from the antibody phage libraries discussed above. Alternatively, Fab′-SH fragments can be directly recovered from E. coli and chemically coupled to form F(ab′)₂ fragments (Carter et al. (1992) Bio/Technology 10:163-167). According to another approach, F(ab′)₂ fragments can be isolated directly from recombinant host cell culture. Other techniques for the production of antibody fragments will be apparent to the skilled practitioner.

Further, any of the previously described anti-CD20 antibodies may be conjugated prior to use in the methods of the present invention. Such conjugated antibodies are available in the art. Thus, the anti-CD20 antibody may be labeled using an indirect labeling or indirect labeling approach. By “indirect labeling” or “indirect labeling approach” is intended that a chelating agent is covalently attached to an antibody and at least one radionuclide is inserted into the chelating agent. See, for example, the chelating agents and radionuclides described in Srivagtava and Mease (1991) Nucl. Med. Bio. 18: 589-603, herein incorporated by reference. Alternatively, the anti-CD20 antibody may be labeled using “direct labeling” or a “direct labeling approach”, where a radionuclide is covalently attached directly to an antibody (typically via an amino acid residue). Preferred radionuclides are provided in Srivagtava and Mease (1991) supra. The indirect labeling approach is particularly preferred. See also, for example, labeled forms of anti-CD20 antibodies described in U.S. Pat. No. 6,015,542, herein incorporated by reference.

The anti-CD20 antibodies are typically provided by standard technique within a pharmaceutically acceptable buffer, for example, sterile saline, sterile buffered water, propylene glycol, combinations of the foregoing, etc. Methods for preparing parenterally administrable agents are described in Remington=s Pharmaceutical Sciences (18^{th} ed.; Mack Pub. Co.: Eaton, Pa., 1990), herein incorporated by reference. See also, for example, WO 98/56418, which describes stabilized antibody pharmaceutical formulations suitable for use in the methods of the present invention.

The following examples are offered by way of illustration and not by way of limitation.

### EXAMPLES

### Example 1

### Initial Clinical Trial

The IL-2 formulation used in this study is manufactured by Chiron Corporation of Emeryville, Calif., under the tradename Proleukin. The IL-2 in this formulation is a recombinantly produced human IL-2 mutein, called aldesleukin, which differs from the native human IL-2 sequence in having the initial alanine residue eliminated and the cysteine residue at position 125 replaced by a serine residue (referred to as des-alanyl-1, serine-125 human interleukin-2). This IL-2 mutein is expressed from E. coli, and subsequently purified by diafiltration and cation exchange chromatography as described in U.S. Pat. No. 4,931,543. The IL-2 formulation marketed as Proleukin is supplied as a sterile, white to off-white preservative-free lyophilized powder in vials containing 1.3 mg of protein (22 MIU).

An initial trial at the University of Rochester used a daily dose of Proleukin combined with weekly Rituxan (Rituximab; IDEC-C2B8; IDEC Pharmaceuticals Corp., San Diego, Calif.) per its package insert dose (375 mg/m² infused over 6 hrs). Proleukin was administered as a daily subcutaneous dose beginning on day 1 through day 5, with the 6 hr Rituxan infusion occurring on day 3. Neither drug was administered on days 6 and 7. The dosing regimen was repeated for 3 additional weeks (i.e., 4 consecutive weeks of treatment). The dose of Proleukin chosen was an intermediate schedule in order to expand the maximum number of NK cells with the fewest side effects. The dose chosen was 4.5 mIU/m² given as a single injection. This dose is approximately equivalent to 900,000 u/m² of Proleukin.

A summary of the data for 5 patients enrolled to date is as follows. Two patients have received the full course of Proleukin. Of the 4 evaluable patients, there have been 2 complete responses (CRs) and 1 partial response (PR). The other evaluable patient has just completed therapy and may be to early in treatment to assess response. These data compare favorably with the reports of Rituxan alone. Response durations are described below. Only one patient has relapsed to date although that subject had had a complete remission.

Of the 5 patients currently undergoing therapy, there have been 2 severe adverse events (SAEs). The 2 SAEs were documented as a pulmonary embolism in a patient who completed therapy (and eventually responded) and a patient who expired from an ill-described CNS event. Only 2 patients have received full, 4-week courses of Proleukin. Thus, the dose in this study appears to be above a strict maximum tolerated dose (MTD) defined in a more typical phase I study.

Several conclusions can be drawn from this limited experience. These two drugs can be administered together, as generally safe, and result in documented responses. The response rate, although higher than expected (the CR rate for Rituximab is 6%) await further study.

### Example 2

### Subsequent Clinical Trial

An open label, single arm study of escalating doses of IL-2 in combination with the labeled dose of Rituximab is carried out. The Rituximab dose is fixed at 375 mg/m² while IL-2 is given in progressively increasing doses until the outpatient MTD is reached. Rituximab is given weekly beginning on week 1 and ending on day 1 of week 4. A daily dose of Proleukin is given starting in week 2 and continuing through week 5. Patients remain on a fixed dose of IL-2 throughout this period. Dose selection for phase II is based on the results obtained in phase I studies.

Treatments Administered

Patients are entered into groups of three. All receive Rituximab 375 mg/m² via 6 hr infusion starting on day 1 and then weekly for 4 weeks (i.e., on days 8, 15, and 22) per the labeled dose for the agent. Proleukin is started in week 2 (on day 8) at the prescribed dose level and given daily by subcutaneous injection for 4 weeks (i.e., through day 35 of the treatment period). Cohorts of 3 patients are treated at that dose and, if tolerated for 2 weeks without a dose limiting toxicity, another cohort of 3 patients enters the study at the next higher dose level. A dose limiting toxicity (DLT) is defined as an adverse reaction that is grade III or higher by National Cancer Institute (NCI) criteria. Some specific criteria that may be encountered during the course of the study include Grade III toxicities (for example, white blood count (a value of 1.0-1.9), platelets (a value of 25-49), hemoglobin (a value of 6.5-7.9), infection (severe, not life threatening), vomiting (6-10 episodes in 24 hours), pulmonary (dyspnea at normal levels of exertion), hypotension (requiring therapy and hospitalization), neurosensory (severe objective sensory loss or paresthesias that interfere with function), neuromoter (objective weakness with impairment of function), fever (oral greater than 39.6-40.4° C.), fatigue (normal activity decreased greater than 50%/inability to work), weight gain (at least 20.0%), local reactions (induration greater than 10 cm²), etc.), and Grade II toxicities (for example, cardiac dysrhythmia (recurrent or persistent but not requiring therapy), cardiac function (decline of resting ejection fraction by more than 20%), cardiac ischemia (asymptomatic ST-T wave changes), and pericardium (pericarditis by clinical criteria). Except for what is listed herein, any grade III toxicity is considered dose limiting.

If a DLT is encountered, 3 additional patients are enrolled at the current dose level. They are treated for the entire 4 weeks of IL-2 therapy. If no further DLTs are encountered, the next dose level is initiated. However if a second DLT is encountered at that dose level, the maximum dose is considered to have been found and the MTD will be the prior dose level.

Once the MTD is determined, an additional 5 patients are treated at that dose. These patients have their blood sampled for Rituxan and IL-2 pharmacokinetics (see the methods described in Maloney et al (1997) Blood 6:2188-2195, Maloney et al. (1997) J Clin. Oncol. 15(10): 3266-3274, and McLaughlin et al. (1998) J Clin. Oncol. 16(8): 2825-2833 for antibody pharmacokinetic sampling and analysis of human anti-chimeric antibody (HACA) and human anti-mouse antibody (HAMA)).

Dose Escalation Scheme

The dose levels for each cohort are given in the table below. The medical monitor will be responsible for assigning the dose to patients entered on study.

| Cohort Number | Dose |
|---|---|
| 1 | 2 × 10⁶ IU qd |
| 2 | 4.5 × 10⁶ IU qd |
| 3 | 7.5 × 10⁶ IU qd |

Three patients are treated at each dose level unless a DLT is encountered in one of those patients. In that case, 3 additional patients are assigned to that dose level per the scheme outlined above. If a patient encounters a DLT, they may continue on study at the prior dose level or be discontinued from the study at the discretion of the investigator and patient. All SAEs require discontinuation and withdrawal from the study.

In order for the dose level to be evaluable, patients must receive 5/7 doses per week or at least 70% of the total dose. Otherwise they are deemed inevaluable and further patients are enrolled at the same dose level until at least 3 have received the evaluable dose or a DLTs reached.

Selection of Study Population

Patients must have histologically confirmed non-Hodgkin lymphoma of low-grade, follicular histology and must not have received prior Rituximab or IL-2. Patients must consent to the combination therapy and must qualify by the criteria given below in order to be included in the study.

Measurements and Efficacy

Of critical importance is the determination of functional expansion of the relevant cells required to enhance the function of Rituximab. Therefore measurements of NK cell number and function, T-cell numbers and function are performed per the schedule outlined below. NK cell expansion is a critical requirement for IL-2's perceived enhancement of Rituximab and will be a component in subsequent dosing decisions. A baseline evaluation (no more than 2 weeks prior to study entry and assignment of dose level) is obtained, during which a number of measurements are made, including tumor measurements, CBC with differential and platelet count, blood chemistries (AST, ALT, bilirubin, creatinine, electrolytes, LDH), urinalysis (protein and blood), TSH, lymphocyte subsets (CD4+, CD8+, CD3-CD56+), and NK cell ADCC function, using standard protocols. A staging evaluation (no more than 4 weeks prior to random assignment to treatments) is obtained, including CT of chest abdomen, pelvis, and EKG, and additional radiological procedures, as indicated. Weekly measurements of creatinine, CBC with differential, and liver function tests and chemistries are obtained. During week 6 or following termination of the study, a physical examination is done, and the following are measured: CBC with differential and platelet count; blood chemistry (AST, ALT, bilirubin, creatinine, electrolytes, LDH), urinalysis protein and blood), lymphocyte subsets (CD4+, CD8+, CD3-CD56+), and TSH.

Efficacy will be assessed in all patients as a secondary variable. An evaluable patient will be defined as: subjects must receive 4 weeks of Rituximab therapy and 70% of the proscribed Proleukin dose and schedule. The response will be evaluated as follows. Tumor measurements will be based upon measurements of perpendicular diameters, using the longest diameter and its greatest perpendicular. Grading of tumor response is as follows:

Complete response-Defined as absence of clinically detectable disease with normalization of any previously abnormal radiographic studies, bone marrow and cerebrospinal fluid (CSF). Response must persist for at least one month. Patients with bone marrow positive for lymphoma prior to chemotherapy must have a repeat biopsy, which is confirmed after a month, negative for lymphoma.

Partial response-Defined as at least 50% decrease in all measurable tumor burden in the absence of new lesions and persisting for at least one month (applicable to measurable tumors only).

Patients are also assessed for effects of Proleukin and Rituximab therapy on the following:

Response duration-Defined as the time from study entry until progressive disease.

Time to progression-Defined as the time from study entry to progressive disease, relapse or death.

Stable disease-Defined as a less than 50% reduction in tumor burden in the absence of progressive disease.

Progressive disease-Defined as representing 25% or greater increase tumor burden or the appearance of a new site of the disease.

Relapse-Defined as the appearance of tumor following documentation of a complete response.

Secondary efficacy evaluations include survival, defined as post-randomization until death, and overall survival, defined as the time from date of diagnosis of NHL until death.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Those skilled in the art will recognize, or be able to ascertain using no more than routine experimentation, many equivalents to the specific embodiments of the invention described herein. Such equivalents are intended to be encompassed by the following claims.

That which is claimed:

1. A method of treating non-Hodgkin's B-cell lymphoma in a mammal, said method comprising concurrent therapy with an anti-CD20 antibody or fragment thereof and interleukin-2 (IL-2) or variant thereof, wherein said concurrent therapy promotes a positive therapeutic response in a treated mammal.

2. The method of claim 1, wherein said mammal is a human.

3. The method of claim 1, wherein said positive therapeutic response is greater than a therapeutic response that would be observed with therapy using said anti-CD20 antibody or fragment thereof alone or with therapy using said IL-2 or variant thereof alone.

4. The method of claim 1, wherein said concurrent therapy comprises administering to said mammal at least one therapeutically effective dose of a pharmaceutical composition comprising said anti-CD20 antibody or fragment thereof and at least one therapeutically effective dose of a pharmaceutical composition comprising said IL-2 or variant thereof.

5. The method of claim 4, wherein said IL-2 or variant thereof is administered subcutaneously.

6. The method of claim 4, wherein said anti-CD20 antibody is an immunologically active chimeric anti-CD20 antibody.

7. The method of claim 6, wherein said chimeric anti-CD20 antibody is IDEC-C2B8.

8. The method of claim 4, wherein said pharmaceutical composition is selected from the group consisting of a stabilized monomeric IL-2 pharmaceutical composition, a multimeric IL-2 composition, a stabilized lyophilized IL-2 pharmaceutical composition, and a stabilized spray-dried IL-2 pharmaceutical composition.

9. The method of claim 8, wherein said IL-2 is recombinantly produced IL-2 having an amino acid sequence for human IL-2 or variant thereof.

10. The method of claim 9, wherein said variant thereof has an amino acid sequence having at least about 70% sequence identity to the amino acid sequence for said human IL-2.

11. The method of claim 8, wherein said anti-CD20 antibody is an immunologically active chimeric anti-CD20 antibody.

12. The method of claim 11, wherein said chimeric anti-CD20 antibody is IDEC-C2B8 or fragment thereof.

13. The method of claim 4, wherein said therapeutically effective dose of said anti-CD20 antibody or fragment thereof is in the range from about 125 mg/m² to about 500 mg/m² and wherein said therapeutically effective dose of IL-2 or variant thereof is in the range from about 2 mIU/m² to about 12 mIU/m².

14. The method of claim 13, wherein said therapeutically effective dose of said anti-CD20 antibody is in the range from about 225 mg/m²to about 400 mg/m² and wherein said therapeutically effective dose of IL-2 or variant thereof is in the range from about 3 mIU/m² to about 6 mIU/m².

15. The method of claim 14, wherein said therapeutically effective dose of said anti-CD20 antibody is about 375 mg/m² and wherein said therapeutically effective dose of IL-2 or variant thereof is about 4.5 mIU/m².

16. The method of claim 4, wherein said concurrent therapy comprises a first administration of said anti-CD20 antibody or fragment thereof on day 1 of a treatment period followed by a first administration of said IL-2 or variant thereof within 7 days of said first administration of said anti-CD20 antibody or fragment thereof to said subject.

17. The method of claim 4, wherein said concurrent therapy comprises multiple dosing of said anti-CD20 antibody or fragment thereof and said IL-2 or variant thereof.

18. The method of claim 17, wherein said multiple dosing comprises administering said anti-CD20 antibody or fragment thereof once per week for a period of 4 weeks starting on day 1 of a treatment period, and administering a daily dose of said IL-2 or variant thereof for a period of 4 weeks starting on day 8 of said treatment period.

19. The method of claim 17, wherein said multiple dosing comprises administering said anti-CD20 antibody or fragment thereof once per week for a period of 4 weeks starting on day 1 of a treatment period, and administering said IL-2 or variant thereof on days 8, 10, 12, 15, 17, 19, 22, 24, 26, and 29 of said treatment period.
